(19) United States
(12) Patent Application Publication
(10) Pub. No.: US 2023/0302000 A1
(43) Pub. Date: Sep. 28, 2023

(54) COMBINATION DRUG TREATMENT TO INCREASE NEUROGENESIS FOR NEUROLOGICAL DISORDERS

(71) Applicant: Progenicyte Therapeutics, Inc., Chicago, IL (US)

(72) Inventor: KIMINOBU SUGAYA, Orlando, FL (US)

(73) Assignee: Progenicyte Therapeutics, Inc., Chicago, IL (US)

(21) Appl. No.: 18/023,314

(22) PCT Filed: Aug. 26, 2021
(86) PCT No.: PCT/US2021/047808

§ 371 Date: Feb. 24, 2023

Related U.S. Application Data
(60) Provisional application No. 63/070,975, filed on Aug. 27, 2020.

(51) Int. Cl. A61K 31/519; A61K 31/405; A61P 25/28

(52) U.S. Cl. CPC A61K 31/519; A61K 31/405; A61P 25/28

(57) ABSTRACT

Neurological disorders involving loss of neurons had been though incurable since the neurons are not regenerate in adults. Disclosed is a combination treatment of MS-818 and phenserine increased neurogenesis under AD pathological conditions. However, any brain damage, including stroke, Alzheimer's disease, ALS, MS, Parkinson's disease, traumatic brain injury, and even aging, is known to increase inflammatory signals (e.g., cytokines). We found those inflammatory signaling increased glial differentiation NSCs. Disclosed is a combination use of MS-818 and non-steroidal anti-inflammatory drug (NSAID) suppress inflammatory signaling increase neurogenesis, indicating this combination of the drugs could be useful as a therapy for any kind of neuronal damages.

### BACKGROUND

Neurological disorders involving loss of neurons had been though incurable since the neurons are not regenerate in adults. However, we learned that we have a stem cell population, called neural stem cells (NSCs), in the brain, which regenerate neurons throughout life. Since the number of NSCs is limited and reduced by aging and diseases, people tried to increase them by transplantation of the cells. In the beginning, NSCs were used but very difficult to find the transplantable cell materials.

### BRIEF DESCRIPTION OF DRAWINGS

FIG. 1 is images of neural cells produced from iPS cells derived from Alzheimer's disease patient and control subject. FIG. 1 A-D show cells derived from Alzheimer's disease patient's iPS cells. FIG. 1 E-H show cells derived from the Control subject's iPS cells. A, E:1 DIV, B, F:7 DIV, C, G: 14 DIV, D, H: 21 DIV. Most of the cells in B, C, and D are astrocytic glial morphology without neuronal morphology (small cell body). Whereas in F, G, and H, both glial and neuronal cells are present.

FIG. 2 is an image of 3D neural cells naturally differentiate from neural stem cells derived from iPS cells in 21 DIV. Neural stem cells (NSCs) still exist in the middle with a compacted cell body. While terminally differentiated cells are observed in the peripheral area of the culture.

FIG. 3 is an image of immuno-cytochemistry of the differentiated neural stem cells derived from control subject iPS cells. Red: GFAP, a marker for astrocytes, Green: beta-3 tubulin, a marker for neurons. Blue: DAPI, counterstaining of nuclei. The cells were differentiated for 21 days, fixed with ice-cold methanol at −20° C. for 15 min, then immune-stained with the antibodies mentioned above. In control, we found both neural and glial differentiation about 50-50%.

FIG. 4 is a graph of the numbers of proliferating cells (NSCs) and newly formed neurons in the 3D neural stem cell culture derived from healthy control subjects and Alzheimer's disease patients, w/wo combination treatment of non-steroidal inflammation drug, 1 μM of Indomethacin and 100 nM of MS-818 (KS-217). Proliferating cells were detected by BrdU incorporation. The cells were incubated with 100 uM of BrdU for 24 hours at 14 DIV, culture 7 more days, then fixed with ice-cold methanol at −20° C. for 15 min. Neurogenesis was detected by cells having both a beta-3 tubulin and a BrdU signal.

### DEFINITIONS

“Anti-inflammatory compounds” as used herein refer to non-steroidal anti-inflammatory compounds (NSAID) such as aspirin, ibuprofen, naproxen, meloxicam, celecoxib, indomethacin, diclofenac, etodolac, fenoprofen, flurbiprofen, meclofenamate, and the like. Other anti-inflammatory compounds include corticosteroid compounds, such as cortisone, prednisone and the like. Other anti-inflammatory compounds include plant derived compounds such as cannabidiol, curcumin, colchicine, resveratrol, capsaicin, epigallocatechin-3-gallate (EGCG), and quercetin. The anti-inflammatory compound includes anti-inflammatory cytokines such as interleukin (IL)-1 receptor antagonist, IL-4, IL-6, IL-10, IL-11, IL-13, or TGF-β. Where appropriate, an anti-inflammatory compound includes the foregoing, or pharmaceutically acceptable salts thereof.

The terms “co-administration”, “co-administered” or “co-administering” as used herein refer to the administration of a substance before, concurrently, or after the administration of another substance such that the biological effects of either substance synergistically overlap.

The term “neurogenesis” as used herein refers to the process by which new neurons are formed in the CNS, namely the brain. Neurogenesis is crucial when an embryo is developing, but also continues in certain brain regions after birth and throughout our lifespan. Adult neurogenesis is the process in which neurons are generated from neural stem cells in the adult. This process differs from prenatal neurogenesis. Neural stem cells (NSCs) are the self-renewing, multipotent cells that generate the main phenotypes of the nervous system.

The term “subject” as used herein refers to an individual. For example, the subject is a mammal, such as a primate, and, more specifically, a human. The term does not denote a particular age or sex. Thus, adult and newborn subjects, whether male or female, are intended to be covered. As used herein, patient or subject may be used interchangeably and can refer to a subject afflicted with a disease or disorder

“Stem cell proliferating pyrimidine compound” or “SCPPC” refers to a pyrimidine compound that induces proliferation of neural stem cells.

The term “MS818” refers to an SCPPC that is a heterocyclic pyrimidine molecule pertaining to the following structure or a pharmaceutically acceptable salt thereof:

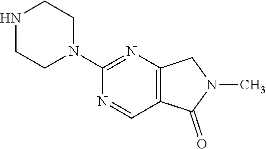 [SMILES: CN1Cc2nc(N3CCNCC3)ncc2C1=O]

The term “pharmaceutically acceptable salt” refers to those salts which retain the biological effectiveness and properties of the active ingredient of the biochemical composition, which are not otherwise undesirable. Pharmaceutically acceptable salts include, but are not limited to, salts formed after combination of the amine compound with inorganic acids like hydrochloric acid, or organic carboxylic acids such as oxalic acid or acetic acid to form oxalate or acetate salts, respectively. Examples of the pharmaceutically acceptable salts of the active agents include hydrochlorides, hydrobromides, sulfates, bisulfites, phosphates, acidic phosphates, acetates, maleates, fumarates, succinates, lactates, tartrates, benzoates, citrates, gluconates, glucanates, methanesulfonates, p-toluenesulfonates and naphthalenesulfonates which are formed from acids capable of forming pharmaceutically acceptable anion-containing nontoxic acid addition salts, hydrates thereof, and quaternary ammonium (or amine) salts or hydrates thereof.

A “pharmaceutically acceptable carrier” may include pharmaceutically-acceptable material, composition or vehicle, such as a liquid or solid filler, diluent, excipient, solvent or encapsulating material, involved in carrying or transporting the disclosed compositions or active agents from one organ, or portion of the body, to another organ, or portion of the body without affecting its biological effect. Each carrier should be “acceptable” in the sense of being compatible with the other ingredients of the composition and not injurious to the subject.

The term “therapeutically effective amount” relates to a dose of the substance that will lead to the desired pharmacological and/or therapeutic effect. The desired pharmacological effect is, to alleviate a condition or disease described herein, or symptoms associated therewith. A therapeutically effective amount of a substance may vary according to factors such as the disease state, age, sex, and weight of the individual, and the ability of the substance to elicit a desired response in the individual. Dosing regimen may be adjusted to provide the optimum therapeutic response. For example, several divided doses may be administered daily, or the dose may be proportionally reduced as indicated by the exigencies of the therapeutic situation.

### DETAILED DESCRIPTION

The present disclosure is based on the previous development of a drug to increase NSCs by peripheral administration (MS-818), but we found the pathological condition of Alzheimer's disease to prevent neurogenesis by increasing glial differentiation of NSCs. It was found that amyloid precursor protein (APP) induced glial differentiation of NSCs, and phenserine suppresses this APP effect. Then, combination treatment of MS-818 and phenserine increased neurogenesis under AD pathological conditions.

Building on this previous work, it is now discovered that any brain damage, including stroke, Alzheimer's disease, ALS, MS, Parkinson's disease, traumatic brain injury, and even aging, is known to increase inflammatory signals (e.g., cytokines) and this inflammatory signaling increased glial differentiation NSCs. Accordingly, provided herein are new compositions and methods that involve a co-administration of MS-818 and non-steroidal anti-inflammatory drug (NSAID) to suppress inflammatory signaling and increase neurogenesis. Accordingly, this combination of the drugs is useful as a therapy to treat neuronal damage from a variety of causes including neurodegenerative disorders and injury.

### Overview

The term MS818 refers to heterocyclic pyrimidine molecule pertaining to the following structure

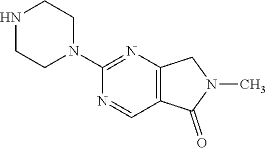 [SMILES: CN1Cc2nc(N3CCNCC3)ncc2C1=O]

or a pharmaceutically acceptable salt thereof. In a specific embodiment, MS818 is:

### 6-Methyl-2-(1-piperazinyl)-6,7-dihydro-5H-pyrrolo[3,4-d]-pyrimidin-5-one maleate

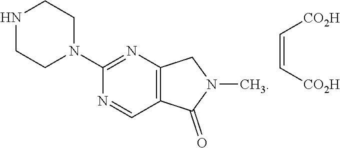

C11-H15-N5-O·C4-H4-O4; Mol wt: 349.35

Also referred in the literature as (2-piperadino-6-methyl-5-oxo-5,6-dihydro-(7H) pyrrolo-[3,4-d] pyrimidine maleate; Mwt. 349.54). Applicants cite to and incorporate herein the teachings of WO2006/133876 (PCT/EP2006/005609) related to pyrimidine based compounds and suitable pharmaceutical salts thereof for stimulating proliferation of stem cells, herein discovered to be stem cell proliferating compounds. The '876 pub sets forth other pyrimidine compounds that may be co-administered with anti-inflammatory compound(s) which include those set forth in the following formula:

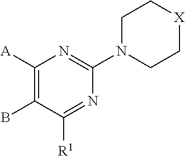

R¹ is H, C₁-C₁₂-alkyl, C₁-C₆-oxyalkyl, C₁-C₆-thioalkyl, C₁-C₆-alkylene-C₁-C₆-oxyalkyl, C₁-C₆-alkylene-CONH₂, CO₁-C₆-alkyl, or C₁-C₆-alkylene-OCO—C₁-C₆-alkyl;
X is —O—, —S—, —CH₂—, >CH—C₁-C₆-alkyl or >NR² wherein R² is H, OH, C₁-C₆-alkyl, C₂-C₆-alkenyl, phenyl, benzyl, CH(phenyl)₂, CO—C₁-C₂₀-alkyl, CO₂—C₁-C₂₀-alkyl, or SOₙ—C₁-C₂₀-alkyl or SOₙ—C₂₀-C₇-aryl (wherein index n is an integer of 0 to 2);
A is H, NH₂. NH—C₁-C₆-alkyl, C₁-C₆-oxyalkyl, or CO₂—C₁-C₆-alkyl;
B is H, CO₂—C₁-C₆-alkyl, CON(C₁-C₁₂-alkyl)₂, C₁-C₆-oxyalkyl, or CH₂CH₂OH; or
A and B together with the carbon atoms to which they are attached form a 5- to 7-membered carbocyclic ring or a heterocyclic ring having N, O or S as the hetero atom;
or a cosmetically acceptable salt thereof;

In specific examples, the stem cell proliferating pyrimidine compounds include those defined by the following formulas:

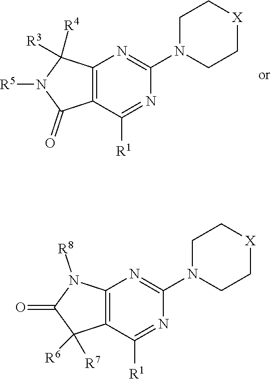

Wherein R1 and X are the same as that defined above and
R¹, R⁴, R⁶ and R⁷ each are independently H, C₁-C₁₂-alkyl, C₁-C₆-oxyalkyl, C₁-C₆-alkylene-C₁-C₆-oxyalkyl, C₁-C₆-alkylene-CONH₂, CO—C₁-C₆-alkyl, or C₁-C₆-alkylene-OCO—C₁-C₆-alky.

Also set forth in the '876 pub are preferred stem cell proliferating pyrimidine compounds that include the following:
2-piperazino-6-methyl-5-oxo-5,6-dihydro(7H)pyrro-[3,4-d]pyrimidine,
2-(4-methylpiperazino)-6-methyl-5-oxo-5,6-dihydro(7H)pyrro-[3,4-d]pyrimidine,
2-(4-ethylpiperazino)-6-methyl-5-oxo-5,6-dihydro(7H)pyrro-[3,4-d]pyrimidine,
2-piperidino-6-methyl-5-oxo-5,6-dihydro(7H)pyrro-[3,4-d]pyrimidine,
2-(4-metylpiperidino)-6-methyl-5-oxo-5,6-dihydro(7H)pyrro-[3,4-d]pyrimidine,
2-(4-ethylpiperidino)-6-methyl-5-oxo-5,6-dihydro(7H)pyrro-[3,4-d]pyrimidine,
2-morpholino-6-methyl-5-oxo-5,6-dihydro(7H)pyrro-[3,4-d]pyrimidine,
2-thiomorpholino-6-methyl-5-oxo-5,6-dihydro(7H)pyrro-[3,4-d]pyrimidine,
2-piperazino-6-ethyl-5-oxo-5,6-dihydro(7H)pyrro-[3,4-d]pyrimidine,
2-piperazino-6-isopropyl-5-oxo-5,6-dihydro(7H)pyrro-[3,4-d]pyrimidine,
2-piperazino-6-n-butyl-5-oxo-5,6-dihydro(7H)pyrro-[3,4-d]pyrimidine,
2-piperazino-6-sec.-butyl-5-oxo-5,6-dihydro(7H)pyrro-[3,4-d]pyrimidine,
2-piperazino-6-tert-butyl-5-oxo-5,6-dihydro(7H)pyrro-[3,4-d]pyrimidine,
2-piperazino-4,6-dimethyl-5-oxo-5,6-dihydro(7H)pyrro-[3,4-d]pyrimidine,
2-piperazino-6,7-dimethyl-5-oxo-5,6-dihydro(7H)pyrro-[3,4-d]pyrimidine,
2-piperazino-6,7,7-trimethyl-5-oxo-5,6-dihydro(7H)pyrro-[3,4-d]pyrimidine,
2-piperidino-4,6-dimethyl-5-oxo-5,6-dihydro(7H)pyrro-[3>4-d]pyrimidine,
2-piperidino-6,7,7-trimethyl-5-oxo-5,6-dihydro(7H)pyrro-[3,4-d]pyrimidine,
2-piperazino-7-methyl-6-ethyl-5-oxo-5,6-dihydro(7H)pyrro-[3,4-d]pyrimidine,
and 2-piperazino-4-methyl-6-ethyl-5-oxo-5,6-dihydro(7H)pyrro-[3,4-d]pyrimidine.

Other compounds include:
2-piperazino-7-methyl-6-oxo-5,6-dihydro(7H)pyrro-[2,3-d]pyrimidine,
2-(4-methylpiperazino)-7-methyl-6-oxo-5,6-dihydro(7H)pyrro-[2,3-d]pyrimidine,
2-(4-ethylpiperazino)-7-methyl-6-oxo-5,6-dihydro(7H)pyrro-[2,3-d]pyrimidine,
2-(4-N-acetylpiperazino)-7-methyl-6-oxo-5,6-dihydro(7H)pyrro-[2,3-d]pyrimidine,
2-(4-N-palmitoylpiperazino)-7-methyl-6-oxo-5,6-dihydro(7H)pyrro-[2,3-d]pyrimidine,
2-(4-N-stearoylpiperazino)-7-methyl-6-oxo-5,6-dihydro(7H)pyrro-[2,3-d]pyrimidine,
2-(4-N-phytanoylpiperazino)-7-methyl-6-oxo-5,6-dihydro(7H)pyrro-[2,3-d]pyrimidine,
2-piperidino-7-methyl-6-oxo-5,6-dihydro(7H)pyrro-[2,3-d]pyrimidine,
2-(4-methylpiperidino)-7-methyl-6-oxo-5,6-dihydro(7H)pyrro-[2,3-d]pyrimidine,
2-(4-etylpiperidino)-7-methyl-6-oxo-5,6-dihydro(7H)pyrro-[2,3-d]pyrimidine,
2-morpholino-7-methyl-6-oxo-5,6-dihydro(7H)pyrro-[2,3-d]pyrimidine,
2-thiomorpholino-7-methyl-6-oxo-5,6-dihydro(7H)pyrro-[2,3-d]pyrimidine,
2-piperidino-7-ethyl-6-oxo-5,6-dihydro(7H)pyrro-[2,3-d]pyrimidine,
2-piperidino-7-n-propyl-6-oxo-5,6-dihydro(7H)pyrro-[2,3-d]pyrimidine,
2-piperidino-7-iso-propyl-6-oxo-5,6-dihydro(7H)pyrro-[2,3-d]pyrimidine,
2-piperidino-7-n-butyl-6-oxo-5,6-dihydro(7H)pyrro-[2,3-d]pyrimidine,
2-piperidino-7-tert-butyl-6-oxo-5,6-dihydro(7H)pyrro-[2,3-d]pyrimidine,
2-piperidino-5-methyl-6-oxo-5,6-dihydro(7H)pyrro-[2,3-d]pyrimidine,
2-piperazino-5-methyl-6-oxo-5,6-dihydro(7H)pyrro-[2,3-d]pyrimidine,
2-piperazino-4,7-dimethyl-6-oxo-5,6-dihydro(7H)pyrro-[2,3-d]pyrimidine,
2-piperidino-5₁7-dimethyl-6-oxo-5,6-dihydro(7H)pyrro-[2,3-d]pyrimidine,
2-piperidino-5,5₁7-trimethyl-6-oxo-5,6-dihydro(7H)pyrro-[2,3-d]pyrimidine,
2-piperazino-5,7-dimethyl-6-oxo-5,6-dihydro(7H)pyrro-[2,3-d]pyrimidine,
2-piperazino-5,7,7-trimethyl-6-oxo-5,6-dihydro(7H)pyrro-[2,3-d]pyrimidine,
2-piperidino-4-methyl-7-ethyl-6-oxo-5,6-dihydro(7H)pyrro-[2,3-d]pyrimidine,
and 2-piperidino-5-methyl-7-ethyl-6-oxo-5,6-dihydro(7H)pyrro-[2,3-d]pyrimidine.

The above stem cell proliferating pyrimidine compounds (SCPPCs) include pharmaceutically acceptable salts thereof, as set forth in the '876 pub.

### Formulations and Administration

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The formulation should suit the mode of administration. The agents of use with the current disclosure can be formulated by known methods for administration to a subject using several routes which include, but are not limited to, parenteral, pulmonary, oral, topical, intradermal, intracranial, intrathecally, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

Administration can be parenteral, pulmonary, oral, topical, intradermal, intramuscular, intraperitoneal, intravenous, subcutaneous, intranasal, epidural, ophthalmic, buccal, or rectal administration. In a specific embodiment, the agents are administered via peripheral intravenous access.

### Neurodegenerative Diseases

Disclosed are new compositions and methods that involve a co-administration of MS-818 and non-steroidal anti-inflammatory drug (NSAID) to suppress inflammatory signaling and increase neurogenesis. Accordingly, this combination of the drugs is useful as a therapy to treat neuronal damage from a variety of causes including neurodegenerative disorders and injury. Such neurological disorders include stroke, Alzheimer's disease, ALS, MS, Parkinson's disease, traumatic brain injury but not limited since neuronal damage associate with inflammation and without controlling such inflammation signals NSCs cannot regenerate neurons.

A stroke is a medical condition in which poor blood flow to the brain causes cell death. There are two main types of stroke: ischemic, due to lack of blood flow, and hemorrhagic, due to bleeding. Both cause parts of the brain to stop functioning properly. Signs and symptoms of a stroke may include an inability to move or feel on one side of the body, problems understanding or speaking, dizziness, or loss of vision to one side. Signs and symptoms often appear soon after the stroke has occurred. If symptoms last less than one or two hours, the stroke is a transient ischemic attack (TIA), also called a mini-stroke. A hemorrhagic stroke may also be associated with a severe headache. The symptoms of a stroke can be permanent. Long-term complications may include pneumonia and loss of bladder control. In one embodiment, a MS-818 and an anti-inflammatory compound are co-administered to a subject recovering from a stroke.

Alzheimer's disease (AD) is a progressive neurodegenerative disorder characterized by cognitive decline, irreversible memory loss, disorientation, and language impairment. AD affects 10% of the population aged greater than 65 and at least 50% of the population aged greater than 85 years. AD has been reported in patients as young as 40-50 years of age, but because the presence of the disease is difficult to detect without histopathological examination of brain tissue, the time of onset in living subjects is unknown. Several etiological factors have been implicated in the pathogenesis of Alzheimer's disease. These factors lead to the activation of a cascade process that brings about neuronal death and serious decline in cognitive function. These bed-ridden patients ultimately succumb to death due to inter-current infections related to aspiration, decubitus and stagnation of urine. In one embodiment, a MS-818 and an anti-inflammatory compound are co-administered to a subject exhibiting symptoms of AD, including for example decrease cognitive function.

Amyotrophic lateral sclerosis, or ALS, is a neurodegenerative neuromuscular disease that results in the progressive loss of motor neurons that control voluntary muscles. The defining feature of ALS is the death of both upper motor neurons (located in the motor cortex of the brain) and lower motor neurons (located in the brainstem and spinal cord). ALS affects the nerve cells that control voluntary muscle movements such as walking and talking. Motor neuron loss continues until the ability to eat, speak, move, and finally the ability to breathe is lost. ALS eventually causes paralysis and early death, usually from respiratory failure. ALS is inherited in 5% to 10% of cases. In most cases, the cause is unknown, though it is predicted to be triggered by environmental factors, such as environmental toxin exposure or smoking. In one embodiment, a MS-818 and an anti-inflammatory compound are co-administered to a subject exhibiting symptoms of ALS.

Multiple sclerosis (MS), also known as encephalomyelitis disseminata, is a demyelinating disease in which the insulating covers of nerve cells in the brain and spinal cord are damaged. This damage disrupts the ability of parts of the nervous system to transmit signals, resulting in a range of signs and symptoms, including physical, mental, and sometimes psychiatric problems. Specific symptoms can include double vision, blindness in one eye, muscle weakness, and trouble with sensation or coordination. MS takes several forms, with new symptoms either occurring in isolated attacks (relapsing forms) or building up over time (progressive forms). Between attacks, symptoms may disappear completely, although permanent neurological problems often remain, especially as the disease advances. Multiple sclerosis is the most common immune-mediated disorder affecting the central nervous system. The cause of MS is unknown; however, it is believed to occur as a result of some combination of genetic and environmental factors such as infectious agents. In one embodiment, a MS-818 and an anti-inflammatory compound are co-administered to a subject exhibiting symptoms of MS, such as muscle spasms.

Parkinson's disease (PD) is a progressive neurodegenerative disease characterized by resting tremors, bradykinesia, muscular rigidity, and postural instability. PD typically develops after the age of 60, though 15% of diagnosed patients are under the age of 50. Family history of PD is an etiological factor for 5-10% of patients diagnosed with the disease, yet only 1% of cases have been shown to be clearly familial. It is estimated that 1.5 million Americans are currently living with PD. In an alternative embodiment, MS-818 and an anti-inflammatory compound are co-administered to a subject exhibiting symptoms of PD. Symptoms of PD include resting tremor, bradykinesia, muscle rigidity, postural instability, freezing of gait, micrographia, “mask-face”, or uncontrolled accelerative movements.

### EXAMPLES

In previous studies, it was found that amyloid precursor protein (APP) induces glial differentiation in NSCs. Further investigations revealed that APP induced cytokines to activate JAK-STAT signaling (1) to induce glial genes. JAK-STAT is working together Notch (2) and BMP/Smad (3) to induce glial differentiation of NSCs. Further, it has been found that MCP-1 and chemokines are also involved in glial differentiation, and APP is upstream of this signaling cascade (4,5). Reduction of APP may reduce this cytokine signaling, reduce glial differentiation of NSCs, and allow NSCs to differentiate into neurons (6).

Without being bound to a particular theory, it is now believed that increased inflammation signaling induces glial differentiation of NSCs, not just APP by itself. Therefore, reducing inflammation of the brain tissue should increase neurogenesis.

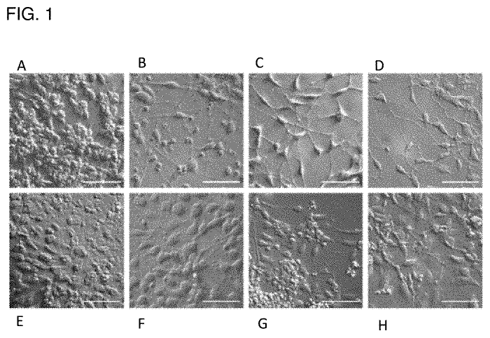

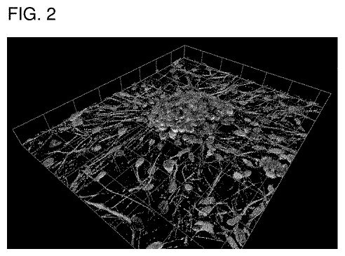

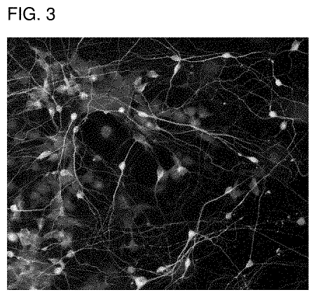

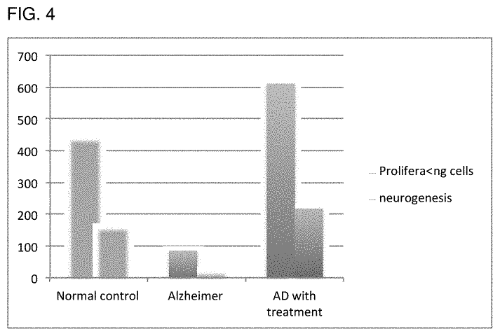

As disclosed herein, neural stem cells derived from AD patient's iPS cells have less neurogenesis compared to the cells derived from the control subject's iPS cells (FIG. 1). A 3D in vitro human brain tissue model was established and was able to keep some NSC population while other cells differentiated into neural cells. (FIG. 2). The differentiated cells in the in vitro 3D human brain model cells derived from control subject iPS cells expressed neuronal and glial markers, indicating both neuronal and glial differentiation (FIG. 3). We tested the proliferation of NSCs and neurogenesis in vitro 3D human brain model. When we count the proliferating cells and newly produced neurons, we found a significant reduction of proliferating NSCs and neurogenesis in AD patients derived cells as compared to the cells derived from control subjects (FIG. 4). The combination treatment of indomethacin (1 μM) and MS-818 (KS-217, 100 nM) increased proliferation and neurogenesis of AD patients derived cells.

These results indicate that we are able to increase neurogenesis in neurological disorders, and improve the brain function, by treating with MS-818 to increase NSCs and NSAID to suppress inflammation and cytokine productions. Neurological disorders including, but not limited to, stroke, Alzheimer's disease, ALS, MS, Parkinson's disease, traumatic brain injury and other neuronal damage associated with inflammation cannot regenerate neurons without controlling such inflammation signals NSC. Thus, the embodiments described herein provide a new therapy to treat any neurological damage.

### REFERENCES

1. Kwak Y D, Dantuma E, Merchant S, Bushnev S, Sugaya K.*: Amyloid-β Precursor Protein Induces Glial Differentiation of Neural Progenitor Cells by Activation of the IL-6/gp130Signaling Pathway. 2011 Neurotoxicity Research. 18(3-4) 328-38
2. Kwak, Y. D., Marutle, A., Dantuma, E., Merchant, S., Bushnev, S. and Sugaya, K* Involvement of notch signaling pathway in amyloid precursor protein induced glial differentiation, 2011 Eur J Pharmacol 650(1) 18-27
3. Young-don Kwak; Brandon James Hendrix and Kiminobu Sugaya*, Secreted type of amyloid precursor protein induces glial differentiation by stimulating the BMP/Smad signaling pathway, BBRC 2014, 447 (3) 394-9
4. Vrotsos E G, Kolattukudy P E, Sugaya K*. MCP-1 involvement in glial differentiation of neuroprogenitor cells through APP signaling. Brain Res Bull. 2009 Apr. 29; 79(2):97-103.
5. Vrotsos E G, Sugaya K*. MCP-1-induced migration of NT2 neuroprogenitor cells involving APP signaling. Cell Mol Neurobiol. 2009 May; 29(3):373-81.
6. Marutle A, Ohmitsu M, Nilbratt N, Greig N H, Nordberg A, Sugaya K*. Modulation of human neural stem cell differentiation in Alzheimer (APP23) transgenic mice by phenserine. Proc Natl Acad Sci USA. 2007 Jul. 24; 104(30):12506-11
7. McGuiness J A, Scheinert R B, Asokan A, et al. Indomethacin Increases Neurogenesis across Age Groups and Improves Delayed Probe Trial Difference Scores in Middle-Aged Rats. Front Aging Neurosci. 2017; 9:280. Published 2017 Sep. 5. doi:10.3389/fnagi.2017.00280
8. Michelle L. Monje, et al., Inflammatory Blockade Restores Adult Hippocampal Neurogenesis Science 302, 1760 (2003); DOI: 10.1126/science.1088417
9. Pamela A. Carpentier and Theo D. Palmer, Immune Influence on Adult Neural Stem Cell Regulation and Function, Neuron. 2009 Oct. 15; 64(1): 79-92
10. Ekdahl, C. T., Claasen, J. H. Bonde, S., Kokaia, Z., and Lindvall, O. (2003). Inflammation is detrimental for neurogenesis in adult brain. Proc. Natl. Acad. Sci. U.S.A 100, 13632-13637. doi: 10.1073/pnas.2234031100
11. Ormerod, B. K., Hanft, S. J., Asokan, A. Haditsch, U., Lee. S. W., and Palmer. T. D. (2013). PPAR gamma activation prevents impairments in spatial memory and neurogenesis following transient illness. Brain Behav. Immun. 29, 28-38. doi: 10.1016/j.bbi.2012.10.017

1. A method of inducing neurogenesis in a subject comprising co-administering a therapeutically effective amount of a stem cell proliferating pyrimidine compound (SCPPC) and a therapeutically effective amount of an anti-inflammatory compound.

2. The method of claim 1, wherein the SCPPC comprises MS818, or a pharmaceutically acceptable salt thereof.

3. The method of claim 1, wherein the anti-inflammatory compound comprises an NSAID.

4. The method of claim 3, wherein the NSAID is selected from the group consisting of aspirin, ibuprofen, naproxen, meloxicam, celecoxib, indomethacin, diclofenac, etodolac, fenoprofen, flurbiprofen, or meclofenamate.

5. The method of claim 3, wherein the NSAID is indomethacin.

6. The method of claim 1, wherein the anti-inflammatory compound comprises a cytokine.

7. The method of claim 6, wherein the cytokine is selected from the group consisting of interleukin (IL)-1 receptor antagonist, IL-4, IL-6, IL-10, IL-11, IL-13, or TGF-β.

8. The method of claim 1, wherein a therapeutically effective amount of an anti-inflammatory compound is an amount that reduces inflammatory signaling in the brain of the subject.

9. The method of claim 1, wherein the therapeutically effective amount of SCPPC induces proliferation of neural stem cells and induces neurogenesis in the brain of the subject.

10. The method of claim 1, wherein co-administering comprises administration of the SCPPC before, concurrently, or after the administration of the anti-inflammatory compound such that the biological effects of either agents overlap.

11. The method of claim 1, wherein the administration comprises peripheral administration.

12. A composition comprising MS818 and an anti-inflammatory compound.

13. The composition of claim 12, wherein the anti-inflammatory compound comprises a NSAID.

14. The composition of claim 13, wherein the NSAID is selected from the group consisting of aspirin, ibuprofen, naproxen, meloxicam, celecoxib, indomethacin, diclofenac, etodolac, fenoprofen, flurbiprofen, or meclofenamate.

15. The composition of claim 13, wherein the NSAID is indomethacin.

16. (canceled)

17. A method of treating impaired brain function comprising co-administering a therapeutically effective amount of a stem cell proliferating pyrimidine compound (SCPPC) and a therapeutically effective amount of an anti-inflammatory compound.
